(19) United States
(12) Patent Application Publication
(10) Pub. No.: US 2024/0352429 A1
(43) Pub. Date: Oct. 24, 2024

(54) AGENT FOR INDUCING VIRAL VECTOR PRODUCTION

(71) Applicant: KANEKA CORPORATION, Osaka (JP)

(72) Inventors: Koki Sasamoto, Hyogo (JP); Takahide Sasaki, Hyogo (JP); Hirofumi Maeda, Hyogo (JP); Mitsuaki Kitano, Hyogo (JP)

(73) Assignee: KANEKA CORPORATION, Osaka (JP)

(21) Appl. No.: 18/623,379

(22) Filed: Apr. 1, 2024

Related U.S. Application Data
(63) Continuation of application No. PCT/JP2022/033743, filed on Sep. 8, 2022.

(30) Foreign Application Priority Data
Oct. 1, 2021 (JP) 2021-162665

(51) Int. Cl. C12N 7/00; C12N 5/00; C12N 15/86

(52) U.S. Cl. CPC C12N 7/00; C12N 5/0018; C12N 15/86; C12N 2500/62; C12N 2501/999; C12N 2750/14143; C12N 2750/14152

(57) ABSTRACT

Described is an agent for inducing viral vector production which includes a cell growth inhibitor A. The cell growth inhibitor A includes a compound which inhibits progression of the cell cycle in G2 phase or M phase. Additionally, a method of producing a viral vector and a method of inducing viral vector production using the same are described. The methods include introducing a nucleic acid into a cell and adding the cell growth inhibitor A to the cell between 6 hours before and 6 hours after the time of introducing the nucleic acid.

### SEQUENCE LISTING

The instant application contains a Sequence Listing which has been submitted electronically in XML format and is hereby incorporated by reference in its entirety. Said XML copy, created on Sep. 6, 2022, is named KC22010.xml and is 39.6 kilobytes in size.

### BACKGROUND

### Technical Field

The present invention relates to an agent for inducing viral vector production.

### Description of Related Art

Viral vectors are widely used in the field of gene therapy. Viral vectors are those in which a gene of a wild-type virus is replaced with a foreign gene, such as a fluorescent protein expression gene or a therapeutic protein expression gene.

With respect to current methods for viral vector production, viral vectors are not sufficiently produced in viral vector production cells. Current methods include introducing a nucleic acid for expression of a viral vector into cells, producing viral vector production cells, and purifying a viral vector from the cell supernatant or cell suspension thereof.

One method of viral vector production uses a histone deacetylase (HDAC) inhibitor as an agent for inducing production (Patent Literature 1). However, this method is not useful since further increases in viral vectors are not observed when the amount of viral vector production (titer) is high in the viral vector production cells.

Additionally, nocodazole-DMSO is disclosed as an AAV production enhancer (Patent Literature 2). However, concentration of nocodazole-DMSO, timing of addition of nocodazole-DMSO, type of solubilizer, and type of nucleic acid to be introduced into a cell are not examined. Furthermore, it is known that nocodazole reduces the productivity of virus-like particles (Non Patent Literature 1).

Accordingly, an agent for inducing viral vector production comprising a compound which inhibits progression of the cell cycle in G2 or M phase and a method of inducing viral vector production using the agent is needed.

### PATENT LITERATURE

Patent Literature 1: JP2020-524498A
Patent Literature 2: WO2020/172624

### Non Patent Literature

Non Patent Literature 1: Applied. Microbiology and Biotechnology 2015 99:9935-9949

One or more embodiments of the present invention is aimed at providing an agent for inducing viral vector production comprising a compound which inhibits progression of the cell cycle in G2 phase or M phase for improving viral vector productivity, a method of producing a viral vector, and a method of inducing viral vector production which use the same agent.

### SUMMARY

The present inventors conducted intensive studies in order to identify an agent for inducing viral vector production comprising cell growth inhibitor A, wherein the cell growth inhibitor A comprises a compound which inhibits progression of the cell cycle in G2 phase or M phase, as well as a method of producing a viral vector and a method of inducing viral vector production which uses the same agent. The methods of producing a viral vector and inducing viral vector production comprise a step of introducing a nucleic acid into a cell and a step of adding cell growth inhibitor A to the cell, wherein the cell growth inhibitor A is added between 6 hours before and 6 hours after the time of introducing the nucleic acid.

### BRIEF DESCRIPTION OF THE DRAWINGS

FIG. 1 illustrates the effect of a final concentration of nocodazole on the titer of adeno-associated virus in Example 1 according to one or more embodiments of the present disclosure.

FIG. 2 illustrates the effect of the timing of addition of cell growth inhibitor A on the titer of adeno-associated virus in Example 2 according to one or more embodiments of the present disclosure.

FIG. 3 illustrates the effect of addition of various cell growth inhibitors A on the titer of adeno-associated virus in Example 3 (Part 1) according to one or more embodiments of the present disclosure.

FIG. 4 illustrates the effect of addition of various cell growth inhibitors A on the titer of adeno-associated virus in Example 3 (Part 2) according to one or more embodiments of the present disclosure.

FIG. 5 illustrates the effect of addition of various cell growth inhibitors A on the titer of adeno-associated virus in Example 3 (Part 3) according to one or more embodiments of the present disclosure.

FIG. 6 illustrates the effect of addition of various cell growth inhibitors A on the titer of adeno-associated virus in Example 3 (Part 4) according to one or more embodiments of the present disclosure.

FIG. 7 illustrates the effect of addition of various cell growth inhibitors A on the titer of adeno-associated virus in Example 3 (Part 5) according to one or more embodiments of the present disclosure.

FIG. 8 illustrates the effect of addition of sodium valproate on the titer of adeno-associated virus in Comparative Example 1 according to one or more embodiments of the present disclosure.

FIG. 9 illustrates the effect of the presence of cell growth inhibitor A on the titer of adeno-associated virus produced using a bioreactor in Example 4 according to one or more embodiments of the present disclosure.

FIG. 10 illustrates the effect of using cell growth inhibitor A and linear covalently closed DNAs on the titer of adeno-associated virus in Example 5 (Part 1) according to one or more embodiments of the present disclosure.

FIG. 11 illustrates the effect of using cell growth inhibitor A and linear covalently closed DNAs on the titer of adeno-associated virus in Example 5 (Part 2) according to one or more embodiments of the present disclosure.

FIG. 12 illustrates a vector map of pAAV-Venus_telRL_ara_TelN in Production Example 2-2 according to one or more embodiments of the present disclosure.

FIG. 13 illustrates a vector map of pRC2-mi342_telRL_ara_TelN in Production Example 2-3 according to one or more embodiments of the present disclosure.

FIG. 14 illustrates a vector map of pHelper_telRL_ara_TelN in Production Example 2-4 according to one or more embodiments of the present disclosure.

FIG. 15 illustrates evaluation results of DNA by electrophoresis in Production Example 2-6 according to one or more embodiments of the present disclosure.

FIG. 16 illustrates evaluation results of DNA by electrophoresis in Production Example 2-7 according to one or more embodiments of the present disclosure.

### DETAILED DESCRIPTION

(Agent for Inducing Viral Vector Production) One or more embodiments of the present disclosure relate to an agent for inducing viral vector production comprising cell growth inhibitor A. In one or more embodiments, the agent further comprises an additional component.

### <Viral Vector>

The viral vector is not particularly limited and may be selected as appropriate depending on the purpose. Examples thereof include an adeno-associated virus (AAV) vector, which has low pathogenicity, adenovirus vector, retrovirus vector, lentivirus vector, herpesvirus vector, poliovirus vector, papillomavirus vector, vaccinia virus vector, poxvirus vector, simian virus vector, and Sendai virus vector.

Examples of the serotype of the AAV include AAV1 (Type 1 AAV), AAV2 (Type 2 AAV), AAV3 (Type 3 AAV), AAV4 (Type 4 AAV), AAV5 (Type 5 AAV), AAV6 (Type 6 Type AAV), AAV7 (Type 7 AAV), AAV8 (Type 8 AAV), AAV9 (Type 9 AAV), AAV10 (Type 10 AAV), AAV11 (Type 11 AAV), AAV12 (Type 12 AAV), AAV13 (Type 13 AAV), AAV14 (Type 14 AAV), and any modified product thereof, but not particularly limited thereto, and may be selected as appropriate depending on the purpose.

The modified product described above is not particularly limited and may be appropriately selected depending on the purpose. Examples thereof include AAV obtained by modification (of wild-type AAV) by genetic recombination, e.g., for improvement of tissue specificity of target cells (tropism of infected cells).

### <Cell Growth Inhibitor A>

The cell growth inhibitor A comprises a compound which inhibits progression of the cell cycle in G2 phase or M phase. The compound may be used as a single kind alone or as a mixture of two or more kinds.

The compound which inhibits progression of the cell cycle in G2 phase or M phase is not particularly limited and may be appropriately selected depending on the purpose. Examples thereof include a compound which inhibits microtubule polymerization and a compound which stabilizes microtubules.

The compound which inhibits microtubule polymerization is not particularly limited and may be selected as appropriate depending on the purpose. Examples thereof include benzimidazole derivative, vinca alkaloid compound, and colchicine derivative.

The benzimidazole derivative is not particularly limited provided it has a benzimidazole ring, and may be selected as appropriate depending on the purpose. Examples thereof include nocodazole, albendazole, mebendazole, thiabendazole, fenbendazole, triclabendazole, flubendazole, oxibendazole, parbendazole, oxfendazole, ricobendazole, or a salt thereof.

The vinca alkaloid compound is not particularly limited and may be selected as appropriate depending on the purpose. Examples thereof include vinblastine, vincristine, vindesine, vinorelbine, vinflunine, or a derivative of said compound or a salt thereof.

The colchicine derivative is not particularly limited and may be selected as appropriate depending on the purpose. Examples thereof include colchicine, colcemid (demecolcine), allocolchicine, thiocolchicine, or a salt thereof.

In one or more embodiments, nocodazole, albendazole, mebendazole, oxibendazole vinblastine, colcemid or a salt thereof is used for inducing viral vector production.

As the compound which inhibits microtubule polymerization, a salt form may be suitably used. Examples of the salt form include hydrochloride and sulfate. Specific examples of the salt form include vinblastine sulfate and vincristine sulfate.

The compound which stabilizes microtubules is not particularly limited and may be selected as appropriate depending on the purpose. Examples thereof include a taxane derivative.

The taxane derivative is not particularly limited provided it has a taxane ring, and may be selected as appropriate depending on the purpose. Examples thereof include paclitaxel and docetaxel.

### <Additional Component>

The additional component described above is not particularly limited and may be selected as appropriate depending on the purpose. Examples thereof include a solubilizer, a surfactant, a colorant, a stabilizer, and a pH adjuster. These components may be used as a single kind alone or as a mixture of two or more kinds thereof.

Examples of the solubilizer include water, an acidic aqueous solution, an alcohol, dimethyl sulfoxide (DMSO), dimethylformamide (DMF), dimethylacetamide (DMA), hexamethylphosphoric triamide (HMPA), acetonitrile, acetone, dioxane, and tetrahydrofuran (THF).

The acidic aqueous solution is not particularly limited and may be selected as appropriate depending on the purpose. Examples thereof include an aqueous solution of an acidic compound, such as hydrochloric acid, sulfuric acid, nitric acid, phosphoric acid, acetic acid, monofluoroacetic acid, difluoroacetic acid, trifluoroacetic acid, formic acid, oxalic acid, malonic acid, or succinic acid. The acidic compound may be used as a single kind alone or as a mixture of two or more kinds thereof.

In one or more embodiments, the concentration of the acidic aqueous solution may be more than 0 and less than the maximum solubility which is unique to the acidic compound. For instance, the concentration of the acidic compound may be more than 0 w/w % and equal to or less than the saturated concentration (w/w %) of the acidic compound at 20° C. and ordinary pressure.

The alcohol is not particularly limited provided it has a hydroxy group, and may be selected as appropriate depending on the purpose. Examples thereof include methanol, ethanol, propanol, isopropanol, n-butyl alcohol, isobutyl alcohol, sec-butyl alcohol, and tert-butyl alcohol.

In one or more embodiments, water, hydrochloric acid, formic acid aqueous solution, ethanol or DMSO are used for inducing viral vector production and solubility of the compound.

### (Method of Producing Viral Vector 1)

In one or more embodiments, the method of producing a viral vector is a method using an agent for inducing viral vector production comprising cell growth inhibitor A.

The method using an agent for inducing viral vector production is not particularly limited and may be selected as appropriate depending on the purpose. Examples thereof include a method of adding the agent for inducing viral vector production to a viral vector production cell.

The viral vector production cell is not particularly limited and may be selected as appropriate depending on the purpose. Examples thereof include those which are obtained by introducing (transfecting) a nucleic acid for expression of a viral vector into a cell.

The cell described above is not particularly limited and may be selected as appropriate depending on the purpose. Examples thereof include a mammalian cell and an insect cell.

The mammalian cell is not particularly limited and may be selected as appropriate depending on the purpose. Examples thereof include a human-derived cell, a mouse-derived cell, a hamster-derived cell, a rat-derived cell, a dog-derived cell, a monkey-derived cell, and a kangaroo-derived cell.

In one or more embodiments, a HEK293 cell, CHO cell, Hela cell, Vero cell, BHK cell, COS cell, MDCK cell, 3T3 cell, HepG2 cell, A549 cell, C2C12 cell, C6 cell, HT-1080 cell, Huh-7 cell, H9C2 cell, HCT116 cell, HT-29 cell, K562 cell, LNCaP cell, Jurkat cell, L6 cell, USO2 cell, or PC12 cell may be used due to the yield of viral vector and safety of the viral vector produced.

The cells described above may include a cell line derived from a parent cell. For example, a HEK293 cell-derived HEK293T cell, a FreeStyle™ 293F cell, or Viral Production Cells 2.0 may be used as the HEK293 cell.

The insect cell is not particularly limited and may be selected as appropriate depending on the purpose. Examples thereof include a moth-derived cell and a fly-derived cell.

In one or more embodiments, a Sf-9 cell, Sf-21 cell, High five cell, Tni cell, or S2 cell may be used.

In one or more embodiments, the cells may include a cell line derived from a parent cell.

The method of culturing a cell is not particularly limited and may be selected as appropriate depending on the purpose. For instance, adhesion culture and suspension culture may be used.

The “nucleic acid” may also be called “polynucleotide”, and examples thereof include deoxyribonucleic acid (DNA) and ribonucleic acid (RNA). The DNA may be a single strand, double strand, or triple strand. The structure of the double-stranded DNA is not particularly limited and may be selected as appropriate depending on the purpose. Examples include a circular plasmid DNA, a linear plasmid DNA, an open circular plasmid DNA, a circular DNA, a linear DNA, and a linear covalently closed DNA.

The “linear covalently closed DNA” may be a linear double-stranded DNA having an end structure which is closed in a hairpin configuration, and may also be abbreviated as “LCC DNA”.

The method of introducing a nucleic acid as described above is not particularly limited and may be selected as appropriate depending on the purpose. Examples thereof include a method using a cationic polymer such as polyethylenimine (PEI), a method using liposome, a method using calcium phosphate, a method using a branched organic compound such as a dendrimer, a method using diethylaminoethyl (DEAE)-dextran, an electroporation method, a particle gun method, a microinjection method, a magnetofection method, and a method using a viral vector.

The timing of addition of the agent for inducing viral vector production is not particularly limited, and may be selected as appropriate depending on the purpose and based on convenience and improvement of the effect of inducing viral vector production. In one or more embodiments, the time of addition is between 6 hours before and 6 hours after the time of introducing the nucleic acid, such as between 4 hours before and 4 hours after the time of introducing the nucleic acid, such as between 5 minutes before and 4 hours after the time of introducing the nucleic acid, such as between 5 minutes before and 2 hours after the time of introducing the nucleic acid, such as between the same time as and 2 hours after the time of introducing the nucleic acid.

The amount of the agent added for inducing viral vector production is not particularly limited, and may be selected as appropriate depending on the purpose. For example, the total amount may be added at one time, or divided into a plurality of doses for the addition.

The method of introducing the nucleic acid is not particularly restricted, and a complex, or mixture, of an introduction reagent (transfection reagent) and the nucleic acid may be formed and then added. Alternatively, an introduction reagent and the nucleic acid may be separately added. The total amount of the introduction reagent and the nucleic acid may also be added at one time, or may be divided into a plurality of doses for the addition.

The time of introducing the nucleic acid refers to the time of adding an introduction reagent (transfection reagent) for introduction of the nucleic acid in a case where the introduction reagent is used. Additionally, the time of introducing the nucleic acid may refer to the time of loading a current, a voltage, or the like in the case of loading of the current, a voltage, or the like for the introduction of the nucleic acid.

Additionally, the time of adding an introduction reagent may refer to the first time of addition in a case where the introduction reagent is divided into multiple doses for the addition.

The lower limit of the final concentration of the cell growth inhibitor A described above is not particularly limited and may be selected as appropriate depending on the purpose. In one or more embodiments, the lower limit may be 1 nM or more, 10 nM or more, 100 nM or more, 200 nM or more, 250 nM or more, 300 nM or more, or 400 nM or more.

The upper limit of the final concentration of the cell growth inhibitor A described above is not particularly limited and may be selected as appropriate depending on the purpose. In one or more embodiments, the upper limit is 100 μM or less, 10 μM or less, 5 μM or less, 2.5 μM or less, 1 μM or less, 800 nM or less, or 600 nM or less.

When the cell growth inhibitor A is nocodazole, the final concentration of nocodazole is not particularly limited, and may be selected as appropriate depending on the purpose. In one or more embodiments, the final concentration of nocodazole is between 100 nM and 5 μM, between 250 nM and 2 μM, between 300 nM and 1 μM, or between 400 nM and 800 nM.

When the cell growth inhibitor A is albendazole, the final concentration of albendazole is not particularly limited, and may be selected as appropriate depending on the purpose. In one or more embodiments the final concentration of albendazole is between 500 nM and 20 μM, or between 1 μM and 10 μM.

When the cell growth inhibitor A is oxibendazole, the final concentration of oxibendazole is not particularly limited, and may be selected as appropriate depending on the purpose. In one or more embodiments, the final concentration of oxibendazole is between 500 nM and 200 μM, between 1 μM and 150 μM, or between 10 μM and 100 μM.

When the cell growth inhibitor A is mebendazole, the final concentration of mebendazole is not particularly limited, and may be selected as appropriate depending on the purpose. In one or more embodiments, the final concentration of mebendazole is between 500 nM and 200 μM, or between 1 μM and 150 μM, between 10 μM and 100 μM.

When the cell growth inhibitor A is vinblastine, the final concentration of vinblastine is not particularly limited, and may be selected as appropriate depending on the purpose. In one or more embodiments, the final concentration of vinblastine is between 10 nM and 10 μM, between 50 nM and 10 μM, or between 100 nM or more and 5 μM.

When the cell growth inhibitor A is colcemid, the final concentration of colcemid is not particularly limited, and may be selected as appropriate depending on the purpose. In one or more embodiments, the final concentration of colcemid is between 10 nM and 10 μM, between 50 nM and 10 μM, or between 100 nM and 5 μM.

### Method of Producing Viral Vector 1

The amount of a viral vector obtained by the method of producing a viral vector 1 is not particularly limited, and may be selected as appropriate depending on the purpose. In one or more embodiments, the titer of the viral vector relative to the titer of a viral vector in the case where the agent for inducing viral vector production is not used may be 1.1-fold or more, 1.2-fold or more, 1.5-fold or more, or 2-fold or more.

The titer of the viral vector is determined by measurement according to a quantitative PCR method (Quantstudio3, SYBR Green method), as follows.

In the quantitative PCR, a material consisting of 12.5 μL of PowerUp™ SYBR® Green Master Mix (Thermo Fisher Scientific K.K.), 0.125 μL of a forward primer (50 μM, SEQ ID NO: 1), 0.125 μL of a reverse primer (50 μM, SEQ ID NO: 2), 11.25 μL of Milli-Q® water, and 1 μL of a reference product or a sample diluted 5000-fold (25 μL in total) is used.

PCR reaction for the above material is carried out by repeating 94° C./15 seconds (heat denaturation), 60° C./30 seconds (annealing), and 72° C./30 seconds (elongation reaction) for 30 cycles using QuantStudio3 (Thermo Fisher Scientific K.K.).

When the viral vector is the adeno-associated virus (AAV) vector, the above reference product to be used is those obtained by subjecting pAAV-MCS Expression Vector (0.67 μg/μL, TE solution, Cell Biolabs, Inc.) to digestion and linearization with PvuII (Takara Bio Inc.) at 37° C. for 2 hours.

The titer of the viral vector in the case where the agent for inducing viral vector production is not used is not particularly limited, and may be selected as appropriate depending on the purpose. In one or more embodiments, the titer is 1×10¹³ vector genomes/L or more, 2×10¹³ vector genomes/L or more, 5×10¹³ vector genomes/L or more, 8×10¹³ vector genomes/L or more, or 1×10¹⁴ vector genomes/L or more.

### Method of Producing Viral Vector 2

In one or more embodiments, the method of producing a viral vector comprises a step of introducing (transfecting) a nucleic acid into a cell and a step of adding cell growth inhibitor A described below to the cell, and may further comprise an additional step.

Step of Introducing (Transfecting) a Nucleic Acid into Cells

The cell is not particularly limited and may be selected as appropriate depending on the purpose. Examples thereof include a mammalian cell and an insect cell.

The mammalian cell is not particularly limited and may be selected as appropriate depending on the purpose. Examples thereof include a human-derived cell, a mouse-derived cell, a hamster-derived cell, a rat-derived cell, a dog-derived cell, a monkey-derived cell, and a kangaroo-derived cell.

In one or more embodiments, a HEK293 cell, CHO cell, Hela cell, Vero cell, BHK cell, COS cell, MDCK cell, 3T3 cell, HepG2 cell, A549 cell, C2C12 cell, C6 cell, HT-1080 cell, Huh-7 cell, H9C2 cell, HCT116 cell, HT-29 cell, K562 cell, LNCaP cell, Jurkat cell, L6 cell, USO2 cell, or PC12 cell may be used, due to the yield of the viral vector and safety of the viral vector produced.

The cell described above may include a cell line derived from a parent cell. For example, a HEK293 cell-derived HEK293T cell, a FreeStyle (trademark) 293F cell, or Viral Production Cells 2.0 may be used as the HEK293 cell.

The insect cell is not particularly limited and may be selected as appropriate depending on the purpose. Examples thereof include a moth-derived cell and a fly-derived cell.

In one or more embodiments, a Sf-9 cell, a Sf-21 cell, a High five cell, a Tni cell, or a S2 cell may be used.

The cell described above may include a cell line derived from a parent cell.

The method of culturing the cell is not particularly limited and may be selected as appropriate depending on the purpose. For example adhesion culture or suspension culture may be used.

The “nucleic acid” described above may also be called “polynucleotide”. Examples thereof include deoxyribonucleic acid (DNA) and ribonucleic acid (RNA). The DNA may be a single strand, double strand, or triple strand. The structure of the double-stranded DNA is not particularly limited and may be selected as appropriate depending on the purpose. Examples include a circular plasmid DNA, a linear plasmid DNA, an open circular plasmid DNA, a circular DNA, a linear DNA, and a linear covalently closed DNA.

The “linear covalently closed DNA” may be a linear double-stranded DNA having an end structure which is closed in a hairpin configuration, and may also be abbreviated as “LCC DNA”.

The method of introducing a nucleic acid is not particularly limited and may be selected as appropriate depending on the purpose. Examples thereof include a method using a cationic polymer, such as polyethylenimine (PEI); a method using liposome; a method using calcium phosphate; a method using a branched organic compound, such as a dendrimer; a method using diethylaminoethyl (DEAE)-dextran; an electroporation method; a particle gun method; a microinjection method; a magnetofection method; and a method using a viral vector.

### Step of Adding Cell Growth Inhibitor A to Cells

The cell growth inhibitor A is not particularly limited and may be selected as appropriate depending on the purpose. Examples thereof include a compound which inhibits progression of cell cycle. The compound may be used as a single kind alone or as a mixture of two or more kinds.

In one or more embodiments, the cell growth inhibitor A does not include a compound which induces cell death, such as sodium valproate.

Examples of the compound which inhibits progression of the cell cycle include a compound which inhibits progression of the cell cycle in G0 phase, a compound which inhibits progression of the cell cycle in G1 phase, a compound which inhibits progression of the cell cycle in S phase, and a compound which inhibits progression of the cell cycle in G2 phase or M phase (corresponding to the cell growth inhibitor A described above).

The compound which inhibits progression of cell cycle in G2 phase or M phase is as described above (agent for inducing viral vector production).

In the step of adding cell growth inhibitor A to a cell, an additional component may be added together with the cell growth inhibitor A. In one or more embodiments, the additional component is as described above (agent for inducing viral vector production).

The timing of addition of the cell growth inhibitor A is not particularly limited provided that it is between 6 hours before and 6 hours after the time of introducing the nucleic acid. The timing may be selected as appropriate depending on the purpose. In one or more embodiments, the timing is between 4 hours before and 4 hours after the time of introducing the nucleic acid, between 5 minutes before and 4 hours after the time of introducing the nucleic acid, between 5 minutes before and 2 hours after the time of introducing the nucleic acid, or between the same time as and 2 hours after the time of introducing the nucleic acid.

The number of additions of the cell growth inhibitor A is not particularly limited, and may be selected as appropriate depending on the purpose. For example, the total amount may be added at one time, or may be divided into a plurality of doses for the addition.

The method of introducing the nucleic acid is not particularly restricted, and a complex, or mixture, of an introduction reagent (transfection reagent) and the nucleic acid may be formed and then added. Alternatively an introduction reagent and the nucleic acid may be separately added. Additionally, the total amount of the introduction reagent and the nucleic acid may also be added at one time, or may be divided into a plurality of doses for the addition.

The time of introducing the nucleic acid may refer to the time of adding an introduction reagent (transfection reagent) for introduction of the nucleic acid in a case where the introduction reagent is used, or may refer to the time of loading a current, a voltage, or the like in the case of loading of the current, a voltage, or the like for the introduction of the nucleic acid.

Additionally, the time of adding an introduction reagent may refer to the first time of addition in a case where the introduction reagent is divided into a plurality of doses for the addition.

The lower limit of the final concentration of the cell growth inhibitor A is not particularly limited, and may be selected as appropriate depending on the purpose. In one or more embodiments the lower limit is 1 nM or more, 10 nM or more, 100 nM or more, 200 nM or more, 250 nM or more, 300 nM or more, or 400 nM or more.

The upper limit of the final concentration of the cell growth inhibitor A is not particularly limited, and may be appropriately selected depending on the purpose. In one or more embodiments, the upper limit is 100 μM or less, 10 μM or less, 5 μM or less, 2.5 μM or less, 1 μM or less, 800 nM or less, or 600 nM or less.

When the cell growth inhibitor A is nocodazole, the final concentration of nocodazole is not particularly limited, and may be appropriately selected depending on the purpose. In one or more embodiments, the final concentration of nocodazole is between 100 nM and 5 μM, between 250 nM and 2 μM, between 300 nM and 1 μM, or between 400 nM and 800 nM.

When the cell growth inhibitor A is albendazole, the final concentration of albendazole is not particularly limited, and may be appropriately selected depending on the purpose. In one or more embodiments, the final concentration of albendazole is between 500 nM and 20 μM, or between 1 μM and 10 μM.

When the cell growth inhibitor A is oxibendazole, the final concentration of oxibendazole is not particularly limited, and may be appropriately selected depending on the purpose. In one or more embodiments, the final concentration of oxibendazole is between 500 nM and 200 μM, between 1 μM and 150 μM, or between 10 μM and 100 μM.

When the cell growth inhibitor A is mebendazole, the final concentration of mebendazole is not particularly limited, and may be appropriately selected depending on the purpose. In one or more embodiments, the final concentration of mebendazole is between 500 nM and 200 μM, between 1 μM and 150 μM, or between 10 μM and 100 μM.

When the cell growth inhibitor A is vinblastine, the final concentration of vinblastine is not particularly limited, and may be appropriately selected depending on the purpose. In one or more embodiments, the final concentration of vinblastine is between 10 nM and 10 μM, between 50 nM and 10 μM, or between 100 nM and 5 μM.

When the cell growth inhibitor A is colcemid, the final concentration of colcemid is not particularly limited, and may be appropriately selected depending on the purpose. In one or more embodiments, the final concentration of colcemid is between 10 nM and 10 μM, between 50 nM and 10 μM, or between 100 nM and 5 μM.

### Additional Step

The additional step is not particularly limited and may be appropriately selected depending on the purpose. Examples of an additional step include a cell culture step before the step of introducing a nucleic acid into a cell and a cell culture step after the step of adding cell growth inhibitor A to a cell.

Cell Culture Step Before the Step of Introducing a Nucleic Acid Into a Cell The cell culture step before the step of introducing a nucleic acid into a cell is not particularly limited and may be appropriately selected depending on the purpose. Examples include a method of culturing a cell at 37° C. in the presence of 8% CO₂ and adjusting the cell number before seeding in a culture vessel.

The cell number to be seeded in the culture vessel is not particularly limited and may be appropriately selected depending on the purpose. In one or more embodiments, the cell number to seeded is between 1×10⁵ cells/mL and 1×10⁹ cells/mL, between 3×10⁵ cells/mL and 1×10⁸ cells/mL, or between 5×10⁵ cells/mL and 1×10⁷ cells/mL.

The volume of culture medium for seeding in the culture vessel is not particularly limited and may be appropriately selected depending on the purpose. In one or more embodiments, the volume of culture medium is between 0.1 mL and 1000 L, between 1 mL and 100 L, between 3 mL and 10 L, or between 5 mL and 1 L.

Cell Culture Step after the Step of Adding Cell Growth Inhibitor A to a Cell

The cell culture step after the step of adding cell growth inhibitor A to a cell is not particularly limited and may be appropriately selected depending on the purpose. Examples include a method of culturing the cell after the addition of the cell growth inhibitor A at 37° C. in the presence of 8% CO₂ and extracting the viral vector from the cell culture containing the cell after the culturing.

The number of days of the culturing is not particularly limited and may be appropriately selected depending on the purpose. In one or more embodiments, the number of days of culturing is between 1 day and 7 days, between 2 days and 6 days, or between 3 days and 5 days.

The amount of the viral vector obtained by the method of producing a viral vector 2 is not particularly limited and may be appropriately selected depending on the purpose. In one or more embodiments, the titer of the viral vector relative to the titer of the viral vector in the case where the cell growth inhibitor A is not added is 1.1-fold or more, 1.2-fold or more, 1.5-fold or more, or 2-fold or more.

The titer of the viral vector is determined by measurement according to quantitative PCR methods.

The quantitative PCR method is as described above (method of producing a viral vector 1).

The titer of the viral vector in the case where the cell growth inhibitor A is not used is not particularly limited and may be appropriately selected depending on the purpose. In one or more embodiments, the titer of the viral vector is 1×10¹³ vector genomes/L or more, 2×10¹³ vector genomes/L or more, 5×10¹³ vector genomes/L or more, 8×10¹³ vector genomes/L or more, or 1×10¹⁴ vector genomes/L or more.

### Method of Inducing Viral Vector Production 1

One aspect of the method of inducing viral vector production is a method using the agent for inducing viral vector production described above comprising cell growth inhibitor A.

The amount of the viral vector obtained by the method of inducing viral vector production 1 is not particularly limited and may be appropriately selected depending on the purpose. In one or more embodiments, the titer of the viral vector relative to the titer of a viral vector in the case where the agent for inducing viral vector production is not used is 1.1-fold or more, 1.2-fold or more, 1.5-fold or more, or 2-fold or more.

The titer of the viral vector may be determined by measurement according to quantitative PCR methods.

The quantitative PCR method is as described above (method of producing a viral vector 1).

The titer of the viral vector in the case where the agent for inducing viral vector production is not used is not particularly limited and may be appropriately selected depending on the purpose. In one or more embodiments, the titer of the viral vector is 1×10¹³ vector genomes/L or more, 2×10¹³ vector genomes/L or more, 5×10¹³ vector genomes/L or more, 8×10¹³ vector genomes/L or more, or 1×10¹⁴ vector genomes/L or more.

The method using the agent for inducing viral vector production is as described above (method of producing a viral vector 1).

### Method of Inducing Viral Vector Production 2

Another aspect of the induction of viral vector production comprises a step of introducing a nucleic acid into a cell, a step of adding cell growth inhibitor A to the cell, and may further comprise an additional step.

The step of introducing a nucleic acid into a cell, the step of adding cell growth inhibitor A to the cell, and the additional step are as described above (method of producing a viral vector 2).

The amount of the viral vector obtained by the method of inducing viral vector production 2 is not particularly limited and may be appropriately selected depending on the purpose. In one or more embodiments, the titer of the viral vector relative to the titer of a viral vector in the case where the cell growth inhibitor A is not added is 1.1-fold or more, 1.2-fold or more, 1.5-fold or more, or 2-fold or more.

The titer of the viral vector may be determined by measurement according to a quantitative PCR method.

The quantitative PCR method is as described above (method of producing a viral vector 1).

The titer of the viral vector in the case where the cell growth inhibitor A is not used is not particularly limited and may be appropriately selected depending on the purpose. In one or more embodiments, the titer of the viral vector is 1×10¹³ vector genomes/L or more, 2×10¹³ vector genomes/L or more, 5×10¹³ vector genomes/L or more, 8×10¹³ vector genomes/L or more, or 1×10¹⁴ vector genomes/L or more.

### EXAMPLES

Hereinafter, examples of one or more embodiments of the present invention are described, but the present invention is not limited to these examples.

### Cell Culture

A suspension HEK293 cell (Thermo Fisher Scientific K.K.; FreeStyle™ 293F cell) was seeded in a culture vessel and cultured by passage every three days or four days at 37° C. in the presence of 8% CO₂.

### Nucleic Acid

### Production Example 1: Double-Stranded Circular Plasmid DNA

pRC2-mi342 (packaging plasmid DNA) and pHelper (helper plasmid DNA) were purchased from Takara Bio Inc. (AAVpro® Helper Free System). pAAV-Venus (vector plasmid DNA) was manufactured by substituting a nucleic acid sequence encoding a green fluorescent protein (GFP) of pAAV-GFP Control Plasmid (Cell Biolabs, Inc.), with a nucleic acid sequence (SEQ ID NO: 3) encoding a fluorescent protein (Venus).

### Production Example 2: Linear Covalently Closed DNA

RC2_LCC_DNA, Helper_LCC_DNA and Venus_LCC_DNA were produced as follows.

In Production Examples 2-2, 2-3, and 2-4 below, a double-stranded circular plasmid DNA used for transformation of E. coli was prepared by introducing a constructed vector into an E. coli DH5α competent cell (9057, Takara Bio Inc.) and culturing and amplifying the resulting transformant. Preparation of a plasmid from a strain retaining the double-stranded circular plasmid DNA was performed with FastGene Plasmid Mini Kit (NIPPON Genetics Co, Ltd.).

### Production Example 2-1: Preparation of Various Genes Used for Preparation of Vector

The nucleic acid sequence (SEQ ID NO: 4) encoding Escherichia virus N15-derived protelomerase (TelN protelomerase) used in the vector construction was prepared by PCR with a synthetic DNA as a template. The synthetic DNA can be produced by, for example, a method of synthesizing a plurality of oligonucleotides designed such that sequences are overlapped, annealing the oligonucleotides, and then elongating them with DNA ligase or DNA polymerase. In one or more embodiments, synthetic DNA may also be obtained by use of the artificial gene synthesis service provided by each company.

The nucleic acid fragment (SEQ ID NO: 5) was totally synthesized as the paired nucleic acid sequences (telRL sequences) which are recognized by protelomerase and was used in the vector construction.

The AraC gene (SEQ ID NO: 6) controlled by a promoter, which was used in the vector construction, was prepared by PCR using a synthetic DNA as a template. The synthetic DNA may be produced by, for example, a method of synthesizing a plurality of oligonucleotides designed such that sequences are overlapped, annealing the oligonucleotides, and then elongating them with DNA ligase or DNA polymerase. In one or more embodiments, synthetic DNA may also be obtained by using the artificial gene synthesis service provided by each company.

The nucleic acid fragment (SEQ ID NO: 7) was totally synthesized as an arabinose inducible promoter which was used in the vector construction.

The nucleic acid sequence (SEQ ID NO: 3) encoding a fluorescent protein (Venus), which was used in the vector construction, was prepared by PCR using a synthetic DNA as a template. The synthetic DNA may be produced by, for example, a method of synthesizing a plurality of oligonucleotides designed such that sequences are overlapped, annealing the oligonucleotides, and then elongating them with DNA ligase or DNA polymerase. In one or more embodiments, synthetic DNA may also be obtained by using the artificial gene synthesis service provided by each company.

The nucleic acid sequence encoding TelN protelomerase was prepared by PCR using Primer 1 (SEQ ID NO: 8, forward primer) and Primer 2 (SEQ ID NO: 9, reverse primer), an AraC gene controlled by a promoter was prepared by PCR using Primer 3 (SEQ ID NO: 10, forward primer) and Primer 4 (SEQ ID NO: 11, reverse primer), and a nucleic acid sequence encoding a fluorescent protein (Venus) was prepared by PCR using Primer 5 (SEQ ID NO: 12, forward primer) and Primer 6 (SEQ ID NO: 13, reverse primer).

Prime STAR MAX DNA Polymerase (Takara Bio Inc.) was used in the PCR, and reaction conditions were in accordance with the method described in the manufacturer's manual.

### Production Example 2-2: Construction of the Vector Having the Gene of Interest

The nucleic acid fragment was prepared by PCR for the nucleic acid sequence (SEQ ID NO: 3) encoding a fluorescent protein (Venus) with Primer 5 (SEQ ID NO: 12) and Primer 6 (SEQ ID NO: 13) and was substituted with the nucleic acid sequence encoding a green fluorescent protein (GFP) of pAAV-GFP Control Plasmid (Cell Biolabs, Inc.) for constructing pAAV-Venus.

Then, the nucleic acid fragment (SEQ ID NO: 5) of a pair of nucleic acid sequences (telRL sequences) recognized by protelomerase was prepared by total synthesis and inserted into PciI site and KasI site of pAAV-Venus for constructing pAAV-Venus_telRL.

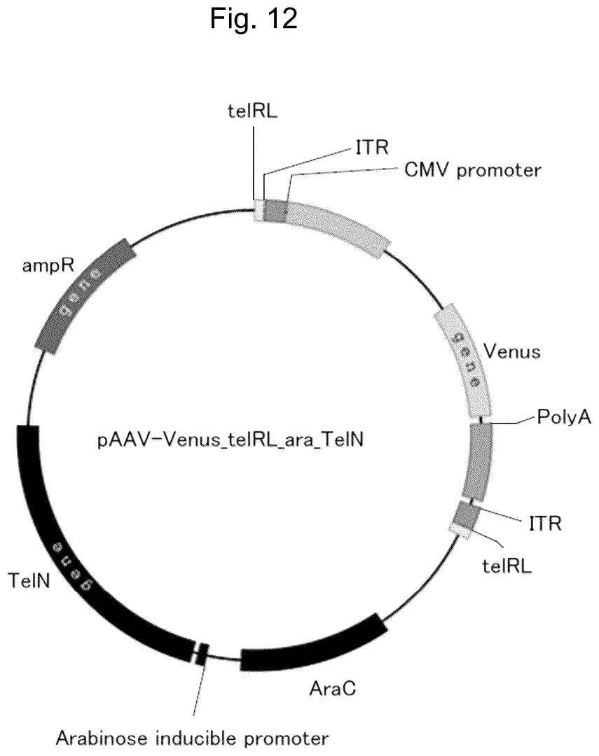

Next, a nucleic acid fragment was prepared by PCR the nucleic acid sequence (SEQ ID NO: 4) encoding TelN protelomerase with Primer 1 (SEQ ID NO: 8) and Primer 2 (SEQ ID NO: 9), and a nucleic acid fragment was prepared by PCR for the AraC gene (SEQ ID NO: 6) under the control of a promoter, with Primer 3 (SEQ ID NO: 10) and Primer 4 (SEQ ID NO: 11), and the nucleic acid fragment (SEQ ID NO: 7) of an arabinose inducible promoter was prepared by total synthesis, and each was inserted into PsiI site of pAAV-Venus_telRL for constructing pAAV-Venus_telRL_ara_TelN (FIG. 12).

The pAAV-Venus_telRL_ara_TelN vector is a vector for production of a linear covalently closed DNA and is designed so that TelN protelomerase is expressed under the control of an arabinose inducible promoter. In addition, telRL sequences are used as a pair of nucleic acid sequences recognized by the protelomerase, and a nucleic acid sequence encoding a fluorescent protein (Venus) is located between the telRL sequences as a nucleic acid sequence (gene of interest) encoding a protein of interest.

The telRL in FIG. 12 is a pair of nucleic acid sequences (telRL sequences) recognized by protelomerase. The ITR in FIG. 12 is an inverted terminal repeat. The CMV Promotor in FIG. 12 is a cytomegalovirus-derived promoter. The Venus in FIG. 12 is a nucleic acid sequence encoding a fluorescent protein (Venus). The PolyA in FIG. 12 is a sequence involved in polyadenylation of messenger RNA. The AraC in FIG. 12 is an AraC gene sequence under the control of a promoter. The Arabinose inducible Promoter in FIG. 12 is a nucleic acid sequence of an arabinose inducible promoter. The TelN in FIG. 12 is a nucleic acid sequence encoding TelN protelomerase. The ampR in FIG. 12 is a nucleic acid sequence encoding an antibiotic-resistant protein (AmpR).

### Production Example 2-3: Construction of the Vector Having a Packaging Gene

The nucleic acid fragment (SEQ ID NO: 5) of a pair of nucleic acid sequences (telRL sequences) recognized by protelomerase was prepared by total synthesis and inserted into EcoRV site and SnaBI site of pRC2-mi342 (Takara Bio Inc.) for constructing pRC2-mi342_telRL.

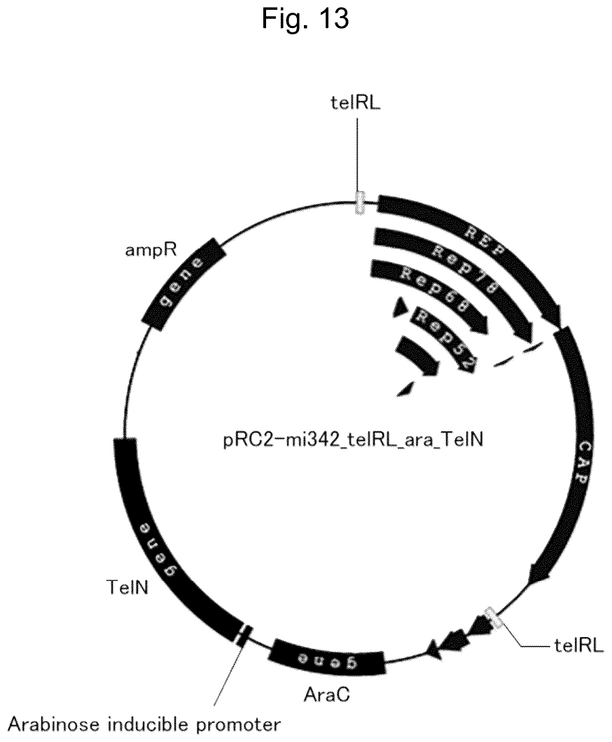

Next, a nucleic acid fragment was prepared by PCR for the nucleic acid sequence (SEQ ID NO: 4) encoding TelN protelomerase, with Primer 1 (SEQ ID NO: 8) and Primer 2 (SEQ ID NO: 9), and a nucleic acid fragment was prepared by PCR for the AraC gene (SEQ ID NO: 6) under the control of a promoter, with Primer 3 (SEQ ID NO: 10) and Primer 4 (SEQ ID NO: 11), and a nucleic acid fragment (SEQ ID NO: 7) of an arabinose inducible promoter was prepared by total synthesis, and each was inserted into SmaI site of pRC2-mi342_telRL for constructing pRC2-mi342_telRL_ara_TelN (FIG. 13).

The pRC2-mi342_telRL_ara_TelN vector is a vector for the production of a linear covalently closed DNA and is designed such that TelN protelomerase is expressed under the control of an arabinose inducible promoter. In addition, telRL sequences are used as a pair of nucleic acid sequences recognized by protelomerase, and the nucleic acid sequences encoding packaging proteins (Rep: SEQ ID NO: 14, Cap: SEQ ID NO: 15) are located between the telRL sequences.

The Rep in FIG. 13 is a nucleic acid sequence encoding the packaging protein Rep of an adeno-associated virus. The Cap in FIG. 13 is a nucleic acid sequence encoding the packaging protein Cap of an adeno-associated virus.

### Production Example 2-4: Construction of the Vector Having a Helper Gene

The nucleic acid fragment (SEQ ID NO: 5) of a pair of nucleic acid sequences (telRL sequences) recognized by protelomerase was prepared by total synthesis and inserted into BamHI site and SalI site of pHelper (Takara Bio Inc.) for constructing pHelper_telRL.

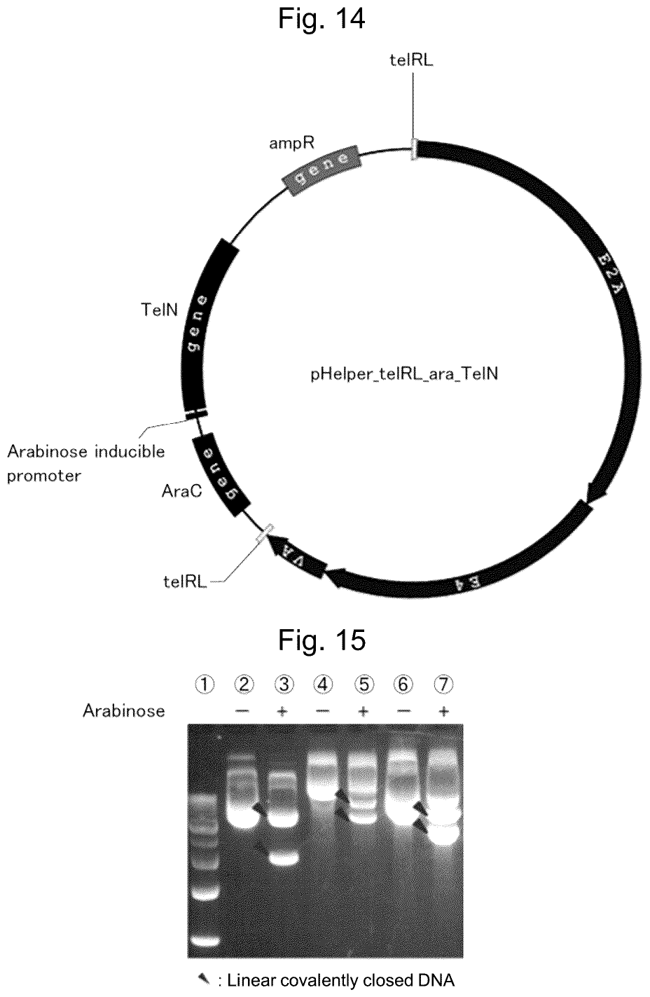

Next, a nucleic acid fragment was prepared by PCR for the nucleic acid sequence (SEQ ID NO: 4) encoding TelN protelomerase, with Primer 1 (SEQ ID NO: 8) and Primer 2 (SEQ ID NO: 9), and a nucleic acid fragment was prepared by PCR for the AraC gene (SEQ ID NO: 6) under the control of a promoter, with Primer 3 (SEQ ID NO: 10) and Primer 4 (SEQ ID NO: 11), and the nucleic acid fragment (SEQ ID NO: 7) of an arabinose inducible promoter was prepared by total synthesis, and each was inserted into NdeI site of pHelper_telRL for constructing pHelper_telRL_ara_TelN (FIG. 14).

The pHelper_telRL_ara_TelN vector is a vector for the production of a linear covalently closed DNA, and is designed such that TelN protelomerase is expressed under the control of an arabinose inducible promoter. In addition, telRL sequences are used as a pair of nucleic acid sequences recognized by protelomerase, and the nucleic acid sequences encoding helper proteins (E2A: SEQ ID NO: 16, E4: SEQ ID NO: 17, VA: SEQ ID NO: 18) are located between the telRL sequences.

The E2A in FIG. 14 is a nucleic acid sequence encoding the helper protein E2A of an adenovirus. The E4 in FIG. 14 is a nucleic acid sequence encoding the helper protein E4 of an adenovirus. The VA in FIG. 14 is a nucleic acid sequence encoding the helper protein VA of an adenovirus.

### Production Example 2-5: Transformation of E. coli

The vector for the production of a linear covalently closed DNA which was constructed in Production Example 2-2, pAAV-Venus_telRL_ara_TelN, the vector for the production of a linear covalently closed DNA which was constructed in Production Example 2-3, pRC2-mi342_telRL_ara_TelN, or the vector for the production of a linear covalently closed DNA which was constructed in Production Example 2-4, pHelper_telRL_ara_TelN, was used for transformation of E. coli as described below.

Twenty-five microliters of a solution of a competent cell of an E. coli NEB100 strain (New England Biolabs, Inc., C3019H) was mixed with a solution of the vector for the production of a linear covalently closed DNA which comprised 100 pg of the pAAV-Venus_telRL_ara_TelN, 100 pg of the pRC2-mi342_telRL_ara_TelN, or 100 pg of the pHelper_telRL_ara_TelN, and the mixture was left to stand on ice for 30 minutes.

The mixture was left to stand for 30 minutes, thereafter heat-treated at 42° C. for 45 seconds, and left to stand on ice for 2 minutes.

The mixture was left to stand for 2 minutes, 225 μL of an SOC culture medium was added thereto, and E. coli was plated to an LB agar culture medium (1% tryptone, 0.5% dry yeast extract, 1% sodium chloride, 0.005% carbenicillin disodium (NACALAI TESQUE, INC.)), a strain to be grown was selected by static culture at 37° C. for 1 day, and E. coli to which the vector for the production of a linear covalently closed DNA was introduced was obtained.

### Production Example 2-6: Culture of Transformed E. coli

The transformed E. coli obtained in Production Example 2-5 was seeded to 2 mL of Plusgrow II culture medium (4% Plusgrow II (NACALAI TESQUE, INC.), 0.005% carbenicillin disodium), cultured with shaking at 37° C. for 6 hours, and then a pre-culture medium was obtained.

One hundred microliters of the pre-culture medium was subjected to subculture in 50 mL of Plusgrow II culture medium (4% Plusgrow II, 0.005% carbenicillin disodium) and was cultured with shaking at 37° C. for 16 hours.

After culturing with shaking for 16 hours, 50 mL of Plusgrow II culture medium (4% Plusgrow II, 0.005% carbenicillin disodium) and 1 mL of an arabinose solution (10% arabinose) were added, followed by culturing with shaking at 37° C. for 1 hour. After culturing with shaking for 1 hour, the bacterial cells were collected by centrifugation.

### Evaluation of the Linear Covalently Closed DNA by Electrophoresis

The linear covalently closed DNA prepared from the E. coli bacteria cell obtained in Production Example 2-6 was evaluated by electrophoresis as described below.

Preparation of DNA from the E. coli bacteria cells was performed using FastGene Plasmid Mini Kit (NIPPON Genetics Co, Ltd.). The resulting DNA solution was applied to wells of 1% agarose gel prepared with TAE buffer and electrophoresed at 100 V for 40 minutes. Staining was performed with a nucleic acid staining agent, and UV light was used for detection. The results are shown in lanes 3, 5, and 7 in FIG. 15.

The evaluation results of DNA obtained from E. coli bacteria cells cultured in the same manner except that 1 mL of the arabinose solution was not added are shown in lanes 2, 4, and 6 in FIG. 15.

In FIG. 15, lane 1 represents DNA marker (1 kb DNA Ladder, 3412A, Takara Bio Inc.), lane 3 represents pAAV-Venus_telRL_ara_TelN-derived sample, lane 5 represents pHelper_telRL_ara_TelN-derived sample, lane 7 represents pRC2-mi342_telRL_ara_TelN-derived sample, lane 2 represents pAAV-Venus_telRL_ara_TelN-derived sample without addition of arabinose solution, lane 4 represents pHelper_telRL_ara_TelN-derived sample without addition of arabinose solution, and lane 6 represents pRC2-mi342_telRL_ara_TelN-derived sample without addition of arabinose solution.

In the results of FIG. 15, two bands derived from the linear covalently closed DNA (as indicated by arrows in FIG. 15, a band derived from a linear covalently closed DNA having the nucleic acid sequence encoding the protein of interest, a band derived from the linear covalently closed DNA having the nucleic acid sequence encoding the packaging protein, or a band derived from the linear covalently closed DNA having the nucleic acid sequence encoding the helper protein, and a band derived from the linear covalently closed DNA having the nucleic acid sequence encoding TelN protelomerase were observed in the E. coli bacteria cells derived from the culture medium with addition of arabinose (lanes 3, 5, and 7), whereas a linear covalently closed DNA-derived band was not observed and only a double-stranded circular plasmid DNA-derived band was observed in the E. coli bacteria cells derived from the culture medium without addition of arabinose (lanes 2, 4, and 6).

These results suggested that when the vector having the nucleic acid sequence encoding protelomerase, the pair of nucleic acid sequences recognized by protelomerase, and the nucleic acid sequence located between the pair of nucleic acid sequences and encoding the protein was introduced into E. coli, TelN protelomerase cleaved and recombined the telRL sequence recognized by TelN protelomerase, and a linear covalently closed DNA having the nucleic acid sequence encoding the protein of interest located between telRL sequences, the nucleic acid sequence encoding the packaging protein located between telRL sequences, or the nucleic acid sequence encoding the helper protein located between telRL sequences was produced.

### Production Example 2-7: Purification of the Linear Covalently Closed DNA

The linear covalently closed DNA obtained in Production Example 2-6 was treated with the restriction enzyme BstZ17I (New England Biolabs, Inc.), and the linear covalently closed DNA was purified with NucleoSpin Gel and PCR Clean-up Kit (MACHEREY-NAGEL GmbH & Co. KG). Thus, a linear covalently closed DNA having a nucleic acid sequence encoding TelN protelomerase and a vector for the production of a linear covalently closed DNA were cleaved at the BstZ17I recognition site thereof.

Next, treatment with Exonuclease (New England Biolabs, Inc.) was performed, and the linear covalently closed DNA was purified with NucleoSpin Gel and PCR Clean-up Kit (MACHEREY-NAGEL GmbH & Co. KG). Thus, the nucleic acid was degraded to nucleotides from the end of the single-stranded or double-stranded DNA.

### Evaluation of Linear Covalently Closed DNA by Electrophoresis

The linear covalently closed DNA obtained in Production Example 2-7 was evaluated by electrophoresis, according to the method described below.

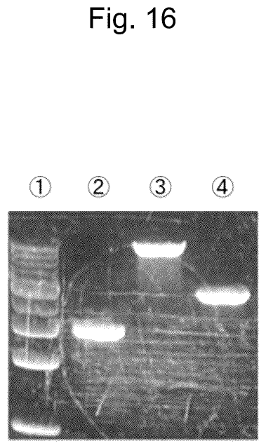

The resulting DNA solution was applied to the wells of 1% agarose gel prepared with TAE buffer and electrophoresed at 100 V for 40 minutes. Staining was performed with a nucleic acid staining agent, and UV light was used for detection. The results are shown in FIG. 16 (lanes 2, 3, and 4).

Herein, a linear covalently closed DNA having a nucleic acid sequence encoding a protein of interest located between telRL sequences is referred to as Venus_LCC_DNA, a linear covalently closed DNA having a nucleic acid sequence encoding a packaging protein located between telRL sequences is referred to as RC2_LCC_DNA, and a linear covalently closed DNA having a nucleic acid sequence encoding a helper protein located between telRL sequences is referred to as Helper_LCC_DNA.

Lane 1 in FIG. 16 represents DNA marker (1 kb DNA Ladder, Takara Bio Inc., 3412A), lane 2 in FIG. 16 represents Venus_LCC_DNA, lane 3 in FIG. 16 represents Helper_LCC_DNA, and lane 4 in FIG. 16 represents RC2_LCC_DNA.

In the results of FIG. 16, a band derived from the linear covalently closed DNA having the nucleic acid sequence encoding the protein located between telRL sequences was observed, whereas bands derived from the linear covalently closed DNA having the nucleic acid sequence encoding TelN protelomerase and derived from the vector for the production of linear covalently closed DNA production were not observed.

These results suggested that the nucleic acid sequence encoding TelN protelomerase was cleaved by the restriction enzyme BstZ17I and the nucleic acid was degraded to nucleotides by Exonuclease from the end of single-stranded or double-stranded DNA. Thus, this result suggested that the ends of the linear covalently closed DNA obtained in Production Example 2-6 were certainly closed.

### Example 1: Influence of the Final Concentration of the Cell Growth Inhibitor A (Containing the Cell Growth Inhibitor A) on the Titer of Adeno-Associated Virus

This Example 1 corresponds to all of the method of producing a viral vector 1, the method of producing a viral vector 2, the method of inducing viral vector production 1, and the method of inducing viral vector production 2.

On the day of introduction of the nucleic acid into cells, suspension HEK293 cells (Thermo Fisher Scientific K.K.; FreeStyle™ 293F cell) were seeded in a 50-mL centrifuge tube equipped with a filter cap, with the number of cells adjusted to 1×10⁶ cells/mL (in an amount of culture medium of 5 mL).

pRC2-mi342 (1 μg/μL, 3 μL, packaging plasmid DNA), pAAV-Venus (1 μg/μL, 1 μL, vector plasmid DNA), pHelper (1 μg/μL, 2 μL, helper plasmid DNA), and PEI MAX (1 μg/μL, 12 μL, Polysciences Inc.) as polyethylenimine (PEI) were mixed and left to stand in Opti-MEM (Thermo Fisher Scientific K.K.) for producing a double-stranded circular plasmid DNA-PEI mixed solution.

Nucleic acids were introduced into the cells by adding the double-stranded circular plasmid DNA-PEI mixed solution to the cell culture medium. Thereafter, AAV2 was produced by culture at 37° C. in the presence of 8% CO₂ for 4 days. In addition to nocodazole (Cayman Chemical Company) as cell growth inhibitor A, DMSO as a solubilizer was used for adjustment to 0 nM, 25 μM, 50 μM, or 200 μM. An amount (50 μL) corresponding to 1% relative to an amount of the cell culture medium of 5 mL was added 4 hours after the introduction of the nucleic acid into the cells so that the final concentration of nocodazole was 0 nM, 250 nM, 500 nM, or 2 μM.

Extraction of the adeno-associated virus from the cell culture medium was carried out as follows.

Four days after the introduction of the nucleic acid into cells, the supernatant and the cell were separated, PEG8000 (Sigma-Aldrich Co. LLC) was added to the supernatant obtained, and the adeno-associated virus vector was concentrated. Triton X-100 (Sigma-Aldrich Co. LLC) was added to the residual for cell lysis, and thereafter KANEKA Endonuclease (KANEKA CORPORATION) was added for degradation of the nucleic acid. EDTA (NIPPON GENE CO., LTD.) was added and centrifuged, and the adeno-associated virus vector was obtained (supernatant-derived sample). Triton X-100 (Sigma-Aldrich Co. LLC) was added to the resulting cell pellets for cell lysis, and thereafter KANEKA Endonuclease (KANEKA CORPORATION) was added for degradation of the nucleic acid. EDTA (NIPPON GENE CO., LTD.) was added and centrifugation was performed, and the adeno-associated virus vector was obtained (cell-derived sample).

Quantification of the adeno-associated virus vector was carried out as follows.

The titer of the adeno-associated virus vector contained in the supernatant-derived sample and the cell-derived sample was measured by a quantitative PCR method (QuantStudio3, SYBR-Green method) with the forward primer (SEQ ID NO: 1) and the reverse primer (SEQ ID NO: 2), as described below.

In the quantitative PCR, a material consisting of 12.5 μL of PowerUp™ SYBR® Green Master Mix (A25742, Thermo Fisher Scientific K.K.), 0.125 μL of the forward primer (50 μM, SEQ ID NO: 1), 0.125 μL of the reverse primer (50 μM, SEQ ID NO: 2), 11.25 μL of Milli-Q® water, and 1 μL of a reference product or a sample diluted 5000-fold (25 μL in total) was used.

PCR reaction for the above material was carried out by repeating 94° C./15 seconds (heat denaturation), 60° C./30 seconds (annealing), and 72° C./30 seconds (elongation reaction) for 30 cycles with QuantStudio3 (Thermo Fisher Scientific K.K.).

The above reference product was produced by digesting pAAV-MCS Expression Vector (0.67 μg/μL, TE solution, Cell Biolabs, Inc.) for linearization with PvuII (1243A, Takara Bio Inc.) at 37° C. for 2 hours.

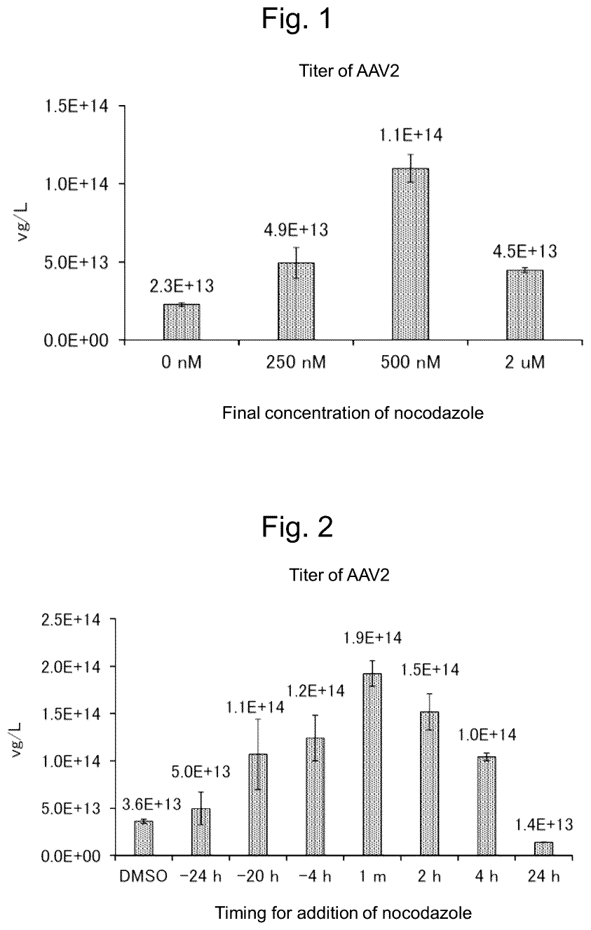

FIG. 1 shows the influence of the final concentration of nocodazole as cell growth inhibitor A on the titer of the adeno-associated virus. FIG. 1 shows the total titer of adeno-associated virus vectors contained in the cell-derived sample and the supernatant-derived sample. In FIG. 1, “0 nM” indicates that the final concentration of nocodazole was 0 nM and that only 50 μL of DMSO was added 4 hours after introduction of the nucleic acid into the cells.

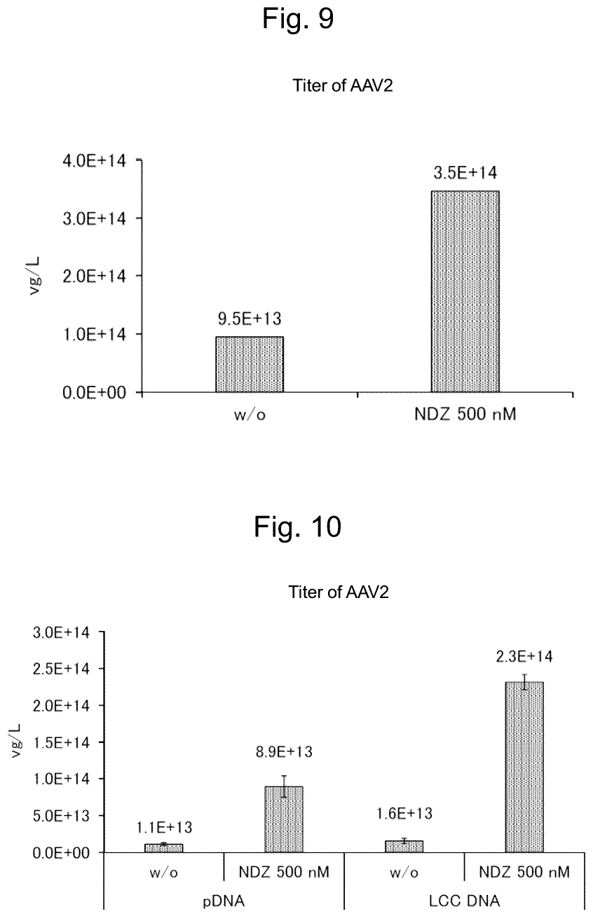

The results shown in FIG. 9 1 indicated that the titer of the adeno-associated virus vector was improved by the addition of the cell culture medium so that the final concentration of nocodazole as cell growth inhibitor A was from 250 nM to 2 μM.

### Example 2: Influence of Timing of Addition of the Cell Growth Inhibitor A (Containing the Cell Growth Inhibitor A) on the Titer of the Adeno Associated Virus

This Example 2 corresponds to all of the method of producing a viral vector 1, the method of producing a viral vector 2, the method of inducing viral vector production 1, and the method of inducing viral vector production 2 (the method of producing a viral vector 2 and the method of inducing viral vector production 2 correspond to the cases of addition of 4 hours before introduction of cell growth inhibitor A into a cell, and 1 minute after, 2 hours after, or 4 hours after introduction of a nucleic acid into a cell).

Nucleic acids were introduced into the cells with the same method as in Example 1, and the adeno-associated virus vector was produced in the conditions where the timing of addition of cell growth inhibitor A was different, using a constant final concentration of cell growth inhibitor A, and the titer was measured. Nocodazole as cell growth inhibitor A and DMSO as a solubilizer were used for adjustment to 50 μM, and an amount (50 μL) corresponding to 1% relative to an amount of the cell culture medium of 5 mL was added 24 hours before, 20 hours before, and 4 hours before introduction of the nucleic acid into the cells, and 1 minute after, 2 hours after, 4 hours after, and 24 hours after introduction of the nucleic acid into the cells so that the final concentration of nocodazole was 500 nM.

FIG. 2 shows the influence of the timing of addition of cell growth inhibitor A on the titer of the adeno-associated virus vector. FIG. 2 shows the titer of the adeno-associated virus vector contained in the cell-derived sample. In FIG. 2, “DMSO” shows that only 50 μL of DMSO was added 1 minute after introduction of the nucleic acid into the cells. “1 m”, “4 h”, and “−4 h”, respectively, show that cell growth inhibitor A was added 1 minute after introduction of the nucleic acid into the cells, 4 hours after introduction of the nucleic acid into the cells, and 4 hours before introduction of the nucleic acid into the cells.

The results shown in FIG. 2 indicated that the titer of the adeno-associated virus vector was improved by the addition of cell growth inhibitor A to the cell culture medium between 24 hours before and 4 hours after the introduction of the nucleic acid into the cells.

### Example 3: Comparison of Various Cell Growth Inhibitors A (Containing the Cell Growth Inhibitor A)

This Example 3 corresponds to all of the method of producing a viral vector 1, the method of producing a viral vector 2, the method of inducing viral vector production 1, and the method of inducing viral vector production 2.

Nucleic acids were introduced into the cells with the same procedure as in Example 1, the adeno-associated virus was produced in the condition where different types of cell growth inhibitor A and a solubilizer were added, and the titer was measured. Various cell growth inhibitors A were adjusted with the solubilizer to the 100-fold concentration of the intended final concentration in the same manner as in Example 1, and an amount (50 μL) corresponding to 1% relative to an amount of the cell culture medium of 5 mL was added 4 hours after introduction of the nucleic acid into the cells.

When the cell growth inhibitor A was nocodazole, DMSO was used as a solubilizer, and the nocodazole was added to cell culture medium so that the final concentration of nocodazole was 500 nM. When the cell growth inhibitor A was fenbendazole (FUJIFILM Wako Pure Chemical Corporation), albendazole (Tokyo Chemical Industry Co., Ltd.), or mebendazole (Tokyo Chemical Industry Co., Ltd.), DMSO was used as a solubilizer, and the cell growth inhibitor A was added to the cell culture medium so that the final concentrations of the cell growth inhibitor A were three kinds of concentration, i.e., 50 μM, 5 μM, and 500 nM. When the cell growth inhibitor A was vinblastine sulfate (FUJIFILM Wako Pure Chemical Corporation), colcemid (Cayman Chemical Company), paclitaxel (FUJIFILM Wako Pure Chemical Corporation), or docetaxel (LKT Laboratories, Inc.), DMSO was used as a solubilizer, and the cell growth inhibitor A was added to the cell culture medium so that the final concentrations of the cell growth inhibitor A were three kinds of concentrations, i.e., 5 μM, 500 nM, and 100 nM. When the cell growth inhibitor A was aphidicolin (FUJIFILM Wako Pure Chemical Corporation) or MG-132 (FUJIFILM Wako Pure Chemical Corporation), DMSO was used as a solubilizer, and the cell growth inhibitor A was added to the cell culture medium so that the final concentrations of the cell growth inhibitor A were three kinds of concentrations, i.e., 100 nM, 10 nM, and 1 nM. When the cell growth inhibitor A was genistein (FUJIFILM Wako Pure Chemical Corporation) or roscovitine (FUJIFILM Wako Pure Chemical Corporation), DMSO was used as a solubilizer, and the cell growth inhibitor A was added to the cell culture medium so that the final concentrations of the cell growth inhibitor A were three kinds of concentrations, i.e., 10 μM, 500 nM, 100 nM. When the cell growth inhibitor A was thymidine (Tokyo Chemical Industry Co., Ltd.), water was used as a solubilizer, and the cell growth inhibitor A was added to the cell culture medium so that the final concentrations of thymidine were three kinds of concentrations, i.e., 10 mM, 1 mM, and 0.1 mM.

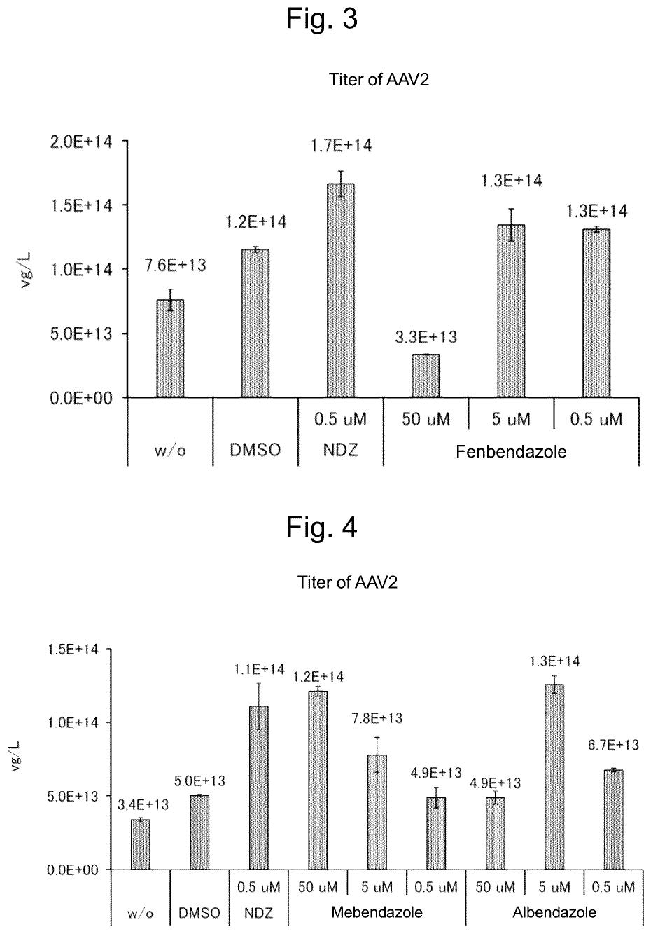

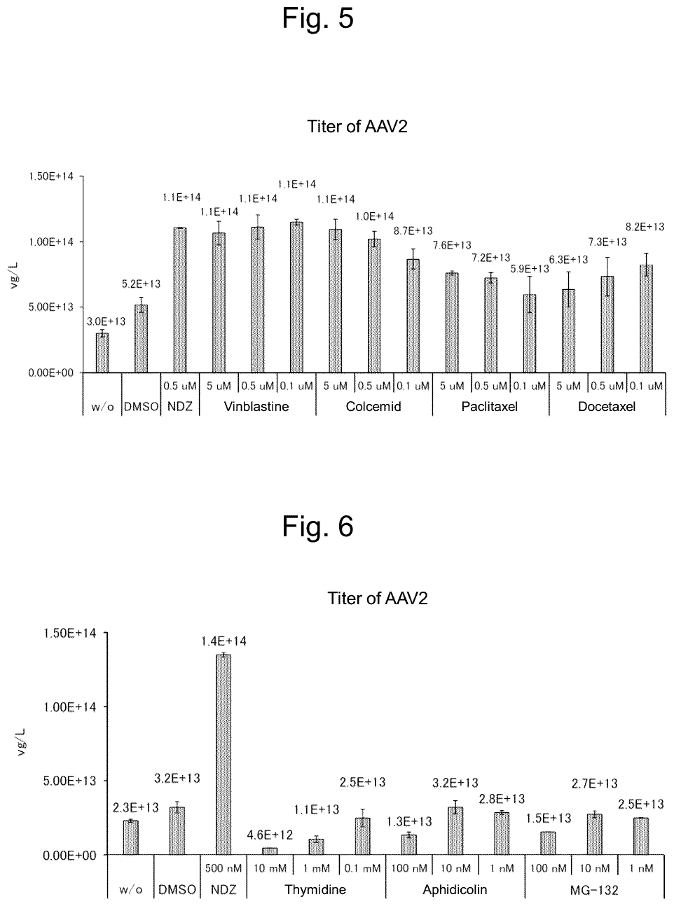

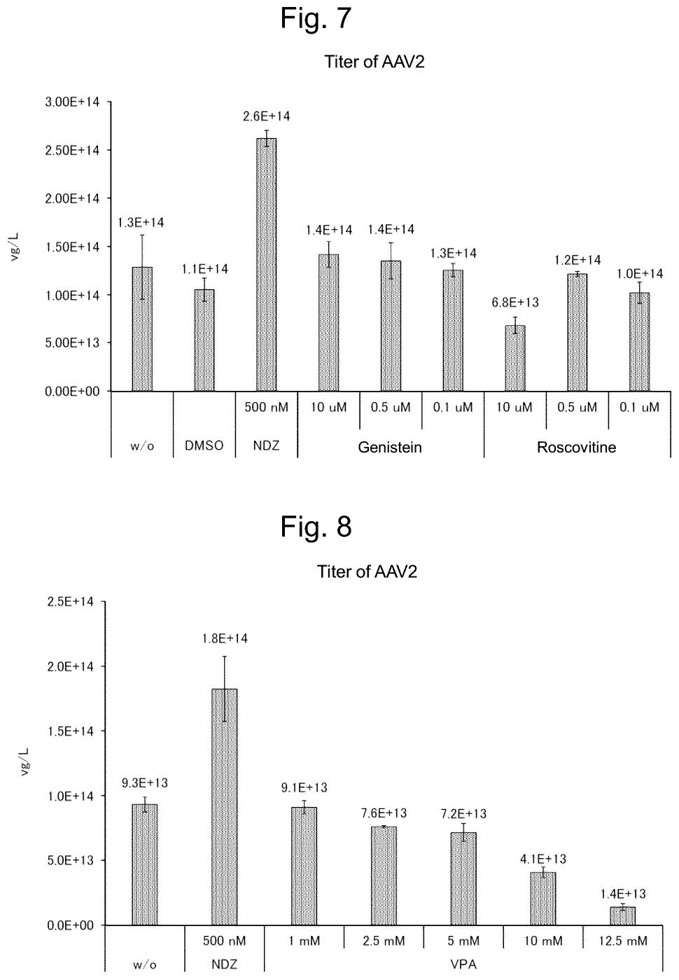

FIG. 3 to FIG. 7 show the influence of addition of various cell growth inhibitors A on the titer of the adeno-associated virus vector. FIGS. 3 to 7 show the titer of the adeno-associated virus vector contained in the cell-derived sample. In FIG. 3 to FIG. 7, “w/o” shows that cell growth inhibitor A and the solubilizer were not added. “DMSO” shows that only 50 μL of DMSO was added 4 hours after introduction of the nucleic acid into the cells. “NDZ” shows that nocodazole was added as cell growth inhibitor A. “VPA” shows that sodium valproate was added as cell growth inhibitor A.

The results shown in FIG. 3 to FIG. 7 indicated that the titer of the adeno-associated virus vector is improved by using not only nocodazole, but also, as cell growth inhibitor A, a benzimidazole derivative such as albendazole, mebendazole, or fenbendazole, a vinca alkaloid compound such as vinblastine, a colchicine derivative such as colcemid, or a taxane derivative such as paclitaxel or docetaxel, serving as a compound inhibiting progression of the cell cycle in G2 phase or M phase and also as a compound inhibiting microtubule polymerization or a compound stabilizing microtubules.

### Comparative Example 1: VPA (Sodium Valproate)

Nucleic acids were introduced into cells with the same procedure as in Example 1, the adeno-associated virus vector was produced in the condition where sodium valproate (VPA, Tokyo Chemical Industry Co., Ltd.) was added as an agent for inducing viral vector production which does not amount to cell growth inhibitor A, and the titer was measured. The sodium valproate was added to the cell culture medium with water as a solubilizer so that the final concentration of the sodium valproate was 1 mM, 2.5 mM, 5 mM, 10 mM, or 12.5 mM. Nocodazole (Cayman Chemical Company), which amounts to cell growth inhibitor A, was used as control. The nocodazole was added with DMSO as a solubilizer so that the final concentration of nocodazole was 500 nM.

FIG. 8 shows the comparison of the influence of addition of cell growth inhibitor A or VPA (sodium valproate) on the titer of the adeno-associated virus vector. FIG. 8 shows the titer of the adeno-associated virus vector contained in the cell-derived sample. In FIG. 8, “w/o” shows that cell growth inhibitor A and a solubilizer were not added. “NDZ” shows that nocodazole was added as cell growth inhibitor A. “VPA” shows that sodium valproate was added. The results shown in FIG. 8 indicated that the effect of improving the titer of the adeno-associated virus vector is larger in an agent for inducing viral vector production comprising cell growth inhibitor A, than an agent for inducing viral vector production comprising sodium valproate (HDAC inhibitor).

### Example 4: Production of the Adeno-Associated Virus Vector with a Bioreactor

This Example 4 corresponds to all of the method of producing a viral vector 1, the method of producing a viral vector 2, the method of inducing viral vector production 1, and the method of inducing viral vector production 2.

On the day of introduction of a nucleic acid into the cells, the suspension HEK293 cells were seeded in a bioreactor (animal cell culture apparatus BCP, volume 1 L, ABLE Corporation), with the number of cells being adjusted to 1×10⁶ cells/mL (in an amount of culture medium of 400 mL).

pRC2-mi342 (1 μg/μL, 240 μL, packaging plasmid DNA), pAAV-Venus (1 μg/μL, 80 μL, vector plasmid DNA), pHelper (1 μg/μL, 160 μL, helper plasmid DNA), and PEI MAX (1 μg/μL, 960 μL, Polysciences Inc.) as polyethylenimine (PEI) were mixed and left to stand in Opti-MEM (Thermo Fisher Scientific K.K.) for production of a double-stranded circular plasmid DNA-PEI mixed solution.

Nucleic acids were introduced into the cells by adding the double-stranded circular plasmid DNA-PEI mixed solution to the cell culture medium. Thereafter, AAV2 was produced by culturing at 37° C. in the presence of 8% CO₂ for 4 days.

Four days after introduction of the nucleic acid into the cells, the supernatant and the cells were separated, PEG8000 (Sigma-Aldrich Co. LLC) was added to the supernatant obtained, and the adeno-associated virus vector was concentrated. Triton X-100 (Sigma-Aldrich Co. LLC) was added to the residual for cell lysis, and thereafter KANEKA Endonuclease (KANEKA CORPORATION) was added for degradation of DNA. EDTA (NIPPON GENE CO., LTD.) was added, centrifugation was performed, and the adeno-associated virus vector was obtained (supernatant-derived sample).

Triton X-100 (Sigma-Aldrich Co. LLC) was added to the resulting cells for cell lysis, and thereafter KANEKA Endonuclease (KANEKA CORPORATION) was added for degradation of nucleic acids. EDTA (NIPPON GENE CO., LTD.) was added, centrifugation was performed, and the adeno-associated virus vector was obtained (cell-derived sample).

Quantification of the adeno-associated virus was performed by the same method as in Example 1.

FIG. 9 shows the influence of the presence of addition of nocodazole as cell growth inhibitor A on the titer of the adeno-associated virus vector produced using the bioreactor. FIG. 9 shows the total titer of adeno-associated virus vector contained in the cell-derived sample and the supernatant-derived sample. In FIG. 9, “w/o” shows that cell growth inhibitor A and a solubilizer were not added. “NDZ 500 nM” shows that adjustment to 50 μM was carried out with nocodazole as cell growth inhibitor A and DMSO as a solubilizer, and an amount (4 mL) corresponding to 1% relative to an amount of the cell culture medium of 400 mL was added 4 hours after introduction of the nucleic acid into the cells such that the final concentration of nocodazole was 500 nM.

The results shown in FIG. 9 indicated that the results of improvement of the titer of the adeno-associated virus vector by cell growth inhibitor A in Examples 1 to 3 were reproduced also at a bioreactor scale, and the adeno-associated virus vector can be produced and induction of the production thereof can be achieved with cell growth inhibitor A.

### Example 5: Production of Adeno-Associated Virus Vector with Cell Growth Inhibitor A (Containing the Cell Growth Inhibitor A) and LCC DNA

This Example 5 corresponds to all of the method of producing a viral vector 1, the method of producing a viral vector 2, the method of inducing viral vector production 1, and the method of inducing viral vector production 2.

On the day of introduction of the nucleic acid into the cells, the suspension HEK293 cells were seeded in a 50-mL centrifuge tube equipped with a filter cap, with the number of cells being adjusted to 1×10⁶ cells/mL (an amount of culture medium of 5 mL).

In the condition where a double-stranded circular plasmid DNA was used as a nucleic acid to be introduced into the cells, pRC2-mi342 (1 μg/μL, 4 μL, packaging plasmid DNA), pAAV-Venus (1 μg/μL, 1 μL, vector plasmid DNA), pHelper (1 μg/μL, 1 μL, helper plasmid DNA), and PEI MAX (1 μg/μL, 12 μL, Polysciences Inc.) as polyethylenimine (PEI) were mixed and left to stand in Opti-MEM (Thermo Fisher Scientific K.K.) for production of a double-stranded circular plasmid DNA-PEI mixed solution. Nucleic acids were introduced into the cells by adding the double-stranded circular plasmid DNA-PEI mixed solution to the cell culture medium. Thereafter, AAV2 was produced by culturing at 37° C. in the presence of 8% CO₂ for 4 days.

In the condition where a linear covalently closed DNA was used as the nucleic acid to be introduced into the cells, RC2_LCC_DNA (1 μg/μL, 4 μL, packaging plasmid DNA), Venus_LCC_DNA (1 μg/μL, 1 μL, vector plasmid DNA), Helper_LCC_DNA (1 μg/μL, 1 μL, helper plasmid DNA), and PEI MAX (1 μg/μL, 12 μL, Polysciences Inc.) as polyethylenimine (PEI) were mixed and left to stand in Opti-MEM (Thermo Fisher Scientific K.K.) for production of a LCC DNA-PEI mixed solution. Nucleic acids were introduced into the cells by adding the LCC DNA-PEI mixed solution to the cell culture medium. Thereafter, AAV2 was produced by culturing at 37° C. in the presence of 8% CO₂ for 4 days.

Extraction and quantification of the adeno-associated virus vector were carried out by the same methods as in Example 1.

FIG. 10 shows the titer of the adeno-associated virus vector contained in the cell-derived sample. In FIG. 10, “pDNA” shows that a double-stranded circular plasmid DNA was used as the nucleic acid to be introduced into the cells, “LCC DNA” shows that a linear covalently closed DNA was used as the nucleic acid to be introduced into the cells, “w/o” shows that cell growth inhibitor A and a solubilizer were not added, and “NDZ 500 nM” shows that nocodazole as cell growth inhibitor A and DMSO as a solubilizer was added 1 minute after introduction of the nucleic acid into the cells by the same method as in Example 2 so that the final concentration of nocodazole was 500 nM.

The cell growth inhibitor A increased the titer of the adeno-associated virus vector eightfold in the condition where a double-stranded circular plasmid DNA was used, whereas it increased the titer fourteenfold in the condition where a linear covalently closed DNA was used, indicating that the effect of the cell growth inhibitor A was remarkably improved under the condition where the linear covalently closed DNA was used. Thus, it was shown that use of the cell growth inhibitor A and a linear covalently closed DNA in combination is favorable for production of an adeno-associated virus vector.

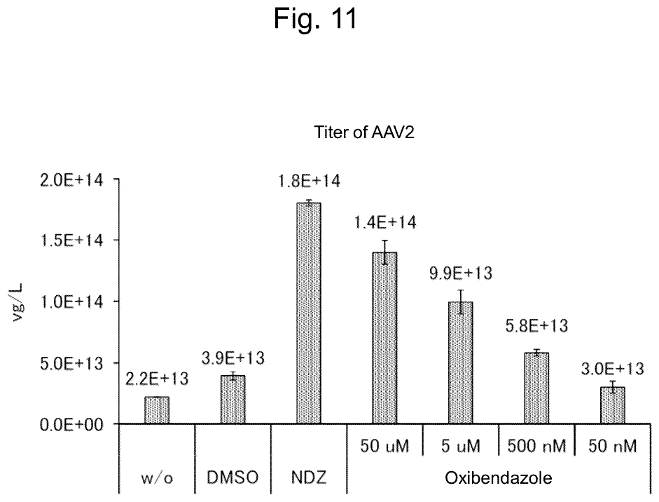

FIG. 11 shows the total titer of the adeno-associated virus vector obtained from the cell-derived sample in the condition where the LCC DNA was used as the nucleic acid to be introduced into the cells. In FIG. 11, “w/o” shows that cell growth inhibitor A and a solubilizer were not added, “DMSO” shows that only 50 μL of DMSO was added 1 minute after introduction of the nucleic acid into the cells. “NDZ” shows that nocodazole as cell growth inhibitor A and DMSO as a solubilizer were added 1 minute after introduction of the nucleic acid into the cells by the same method as in Example 2 such that the final concentration of nocodazole was 500 nM. When the cell growth inhibitor A was oxibendazole (Tokyo Chemical Industry Co., Ltd.), DMSO was used as a solubilizer, and oxibendazole was added to the cell culture medium so that the final concentrations of oxibendazole were four kinds of concentrations, i.e., 50 μM, 5 μM, 500 nM, and 50 nM. The titer of the adeno-associated virus vector was increased not only in the condition where nocodazole was used, but also in the condition where oxibendazole was used, indicating that the cell growth inhibitor A which is favorable to be used in combination with a linear covalently closed DNA for the production of an adeno-associated virus is not limited to nocodazole.

### Example 6: Production of Adeno-Associated Virus Vector with Cell Growth Inhibitor A (Containing the Cell Growth Inhibitor A) and a Solubilizer Other Than DMSO

Example 6 corresponds to all of the method of producing a viral vector 1, the method of producing a viral vector 2, the method of inducing viral vector production 1, and the method of inducing viral vector production 2.

On the day of introduction of the nucleic acid into the cell, the suspension HEK293 cells were seeded in a 50-mL centrifuge tube equipped with a filter cap, with the number of cells being adjusted to 1×10⁶ cells/mL (an amount of culture medium of 5 mL).

pRC2-mi342 (1 μg/μL, 4 μL, packaging plasmid DNA), pAAV-Venus (1 μg/μL, 1 μL, vector plasmid DNA), pHelper (1 μg/μL, 1 μL, helper plasmid DNA), and PEI MAX (1 μg/μL, 12 μL, Polysciences Inc.) as polyethylenimine (PEI) were mixed and left to stand in Opti-MEM (Thermo Fisher Scientific K.K.) for production of a double-stranded circular plasmid DNA-PEI mixed solution.

Nucleic acids were introduced into the cells by adding the double-stranded circular plasmid DNA-PEI mixed solution to the cell culture medium. Thereafter, AAV2 was produced, or the production thereof was induced by culturing at 37° C. in the presence of 8% CO₂ for 4 days. Nocodazole (Cayman Chemical Company) as cell growth inhibitor A, and hydrochloric acid, formic acid aqueous solution or ethanol as a solubilizer were added to the cell culture medium 1 minute after introduction of the nucleic acid into the cells so that the final concentration of nocodazole was 500 nM.

Extraction and quantification of the adeno-associated virus vector were carried out by the same methods as in Example 1.

One or more embodiments of the present invention include an agent for inducing viral vector production comprising cell growth inhibitor A, wherein the cell growth inhibitor A comprises a compound which inhibits progression of the cell cycle in G2 phase or M phase.

In another aspect, the compound which inhibits progression of cell cycle in G2 phase or M phase comprises a compound which inhibits microtubule polymerization or a compound which stabilizes microtubules.

In another aspect, the compound which inhibits progression of the cell cycle in G2 phase or M phase comprises a benzimidazole derivative, a vinca alkaloid compound, or a colchicine derivative.

In another aspect, the compound which inhibits progression of the cell cycle in G2 phase or M phase comprises at least one selected from nocodazole, albendazole, mebendazole, vinblastine, colcemid, thiabendazole, fenbendazole, triclabendazole, flubendazole, oxibendazole, parbendazole, paclitaxel, and docetaxel.

In another aspect, the agent comprises water, hydrochloric acid, formic acid aqueous solution, ethanol, or DMSO as a solubilizer.

In another aspect, the viral vector is an adeno-associated virus vector.

One or more embodiments of the present invention also include a method of producing a viral vector, wherein the method uses the agent for inducing viral vector production.

Additionally, one or more embodiments of the present invention include a method of producing a viral vector comprising a step of introducing a nucleic acid into a cell, and a step of adding cell growth inhibitor A to the cell, wherein the cell growth inhibitor A is added between 6 hours before and 6 hours after the time of introducing the nucleic acid.

In another aspect, the cell growth inhibitor A, and water, hydrochloric acid, formic acid aqueous solution, ethanol, or DMSO as a solubilizer are added in the step of adding cell growth inhibitor A to the cell.

In another aspect, the viral vector is an adeno-associated virus vector.

In another aspect, the nucleic acid is a linear covalently closed DNA.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

The present international application claims the priority based on Japanese Patent Application No. 2021-162665 filed on Oct. 1, 2021, and the entire content of Japanese Patent Application No. 2021-162665 is incorporated into the present international application.

What is claimed is:

1. A method of producing a viral vector, comprising:
an introducing step of introducing a linear covalently closed DNA to cells,
an adding step of adding a compound which inhibits progression of the cell cycle in G2 phase or M phase to the cells, and
a culturing step of culturing the cells after the introducing step and the adding step.

2. The method of claim 1, wherein the adding step is performed between 6 hours before and 6 hours after the introduction step.

3. The method of claim 1, wherein water, hydrochloric acid, formic acid aqueous solution, ethanol, and/or DMSO are further added to the cells as a solubilizer in the adding step.

4. The method of claim 2, wherein the viral vector is an adeno-associated virus vector.

5. A method of inducing viral vector production, comprising:
an adding step of adding a compound which inhibits progression of the cell cycle in G2 phase or M phase to cells to which introduction of a linear covalently closed DNA is carried out, and
a culturing step of culturing the cells after the adding step and the introduction.

6. The method of claim 5, wherein the adding step is performed between 6 hours before and 6 hours after the introduction.

7. The method of claim 5, wherein the viral vector is an adeno-associated virus.

8. The method of claim 1, wherein the compound which inhibits progression of the cell cycle in G2 phase or M phase inhibits microtubule polymerization or stabilizes microtubules.

9. The method of claim 1, wherein the compound which inhibits progression of the cell cycle in G2 phase or M phase comprises a benzimidazole derivative, a vinca alkaloid compound, and/or a colchicine derivative.

10. The method of claim 1, wherein the compound which inhibits progression of the cell cycle in G2 phase or M phase is selected from the group consisting of nocodazole, albendazole, mebendazole, vinblastine, colcemid, thiabendazole, fenbendazole, triclabendazole, flubendazole, oxibendazole, parbendazole, paclitaxel, and docetaxel.

11. The method of claim 1, wherein the linear covalently closed DNA further comprises a nucleic acid sequence encoding a protein of interest and/or an inverted terminal repeat.

12. The method of claim 1, wherein the introducing step further introduces a linear covalently closed DNA encoding a helper protein and/or a packaging protein to the cells.

13. The method of claim 5, wherein the linear covalently closed DNA further comprises a nucleic acid sequence encoding a protein of interest and/or an inverted terminal repeat.


SEQUENCE LISTING (18 sequences)

SEQ ID NO: 1 - DNA, 20 bp, synthetic construct
  misc_feature 1..20: note = Primer Fw
  source 1..20: mol_type = other DNA
tgggttcaagcgattctcct

SEQ ID NO: 2 - DNA, 21 bp, synthetic construct
  misc_feature 1..21: note = Primer Re
  source 1..21: mol_type = other DNA
cctgtaatcccagcaatttgg

SEQ ID NO: 3 - DNA, 720 bp, synthetic construct
  misc_feature 1..720: note = Venus
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 4 - DNA, 1896 bp, Escherichia virus N15
  misc_feature 1..1896: note = TelN
  source 1..1896: mol_type = genomic DNA
atgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaa

SEQ ID NO: 5 - DNA, 56 bp, synthetic construct
  misc_feature 1..56: note = telRL
  source 1..56: mol_type = other DNA
tatcagcacacaattgcccattatacgcgcgtataatggactattgtgtgctgata

SEQ ID NO: 6 - DNA, 1143 bp, synthetic construct
  misc_feature 1..1143: note = AraC
  source 1..1143: mol_type = other DNA
gctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 7 - DNA, 55 bp, synthetic construct
  misc_feature 1..55: note = promoter
  source 1..55: mol_type = other DNA
tacctgacgctttttatcgcaactctctactgtttctccatacccgtttttttgg

SEQ ID NO: 8 - DNA, 24 bp, synthetic construct
  misc_feature 1..24: note = TelN Fw
  source 1..24: mol_type = other DNA
atgagcaaggtaaaaatcggtgag

SEQ ID NO: 9 - DNA, 23 bp, synthetic construct
  misc_feature 1..23: note = TelN Re
  source 1..23: mol_type = other DNA
ttagctgtagtacgtttcccatg

SEQ ID NO: 10 - DNA, 27 bp, synthetic construct
  misc_feature 1..27: note = AraC Fw
  source 1..27: mol_type = other DNA
gctaatcttatggataaaaatgctatg

SEQ ID NO: 11 - DNA, 24 bp, synthetic construct
  misc_feature 1..24: note = AraC Re
  source 1..24: mol_type = other DNA
ttatgacaacttgacggctacatc

SEQ ID NO: 12 - DNA, 19 bp, synthetic construct
  misc_feature 1..19: note = Venus Fw
  source 1..19: mol_type = other DNA
atggtgagcaagggcgagg

SEQ ID NO: 13 - DNA, 22 bp, synthetic construct
  misc_feature 1..22: note = Venus Re
  source 1..22: mol_type = other DNA
ttacttgtacagctcgtccatg

SEQ ID NO: 14 - DNA, 1866 bp, Adeno associated virus
  misc_feature 1..1866: note = Rep
  source 1..1866: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 15 - DNA, 2208 bp, Adeno associated virus
  misc_feature 1..2208: note = Cap
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 16 - DNA, 5336 bp, Adenovirus
  misc_feature 1..5336: note = E2A
  source 1..5336: mol_type = genomic DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcg

SEQ ID NO: 17 - DNA, 3201 bp, Adenovirus
  misc_feature 1..3201: note = E4
  source 1..3201: mol_type = genomic DNA
cccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaa

SEQ ID NO: 18 - DNA, 743 bp, Adenovirus
  misc_feature 1..743: note = VA
  source 1..743: mol_type = genomic DNA
tcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacg